(19) United States
(12) Patent Application Publication
(10) Pub. No.: US 2025/0205006 A1
(43) Pub. Date: Jun. 26, 2025

(54) Intelligent Code Cart

(71) Applicants: Perry Brunette, Irvine, CA (US); Lianna Li Zhang, Irvine, CA (US)

(72) Inventors: Perry Brunette, Irvine, CA (US); Lianna Li Zhang, Irvine, CA (US)

(73) Assignee: PathFree Technologies Corporation, Irvine, CA (US)

(21) Appl. No.: 18/990,981

(22) Filed: Dec. 20, 2024

Related U.S. Application Data
(60) Provisional application No. 63/613,310, filed on Dec. 21, 2023.

(51) Int. Cl. A61B 50/13; A61B 34/00; A61B 50/18; A61B 90/00; A61B 90/98; A61G 12/00; G16H 40/63; G16H 70/20

(52) U.S. Cl. CPC A61B 50/13; A61B 34/25; A61B 90/98; A61G 12/001; G16H 40/63; G16H 70/20; A61B 2034/252; A61B 2050/185; A61B 2090/3612

(57) ABSTRACT

The intelligent code cart provides assistance and guidance to a response team in performing cardiac pulmonary resuscitation. The intelligent code cart can provide graphical and audio instructions to the team based on the ACLS guidelines and also warn the team in case any errors or omissions are observed in following the ACLS protocol. The intelligent code cart comprises a cart, a control unit, a display, one or more cameras, a speaker and input devices. The drawers of the cart can open or provide access to the tools and medication as needed while maintaining a secure environment for other areas. The monitors record and observe while the display can provide guidance on treatment and can be linked to a historical database, to hospitals, records and other resources for guidance or handoff to other medical personnel.

### CROSS-REFERENCE TO RELATED APPLICATIONS

This application claims the benefit of Provisional Application Ser. No. 63/613,310 filed Dec. 21, 2023, the entire contents of which is hereby expressly incorporated by reference herein.

### PRIOR ART

An emergency medical condition means a sudden and, at the time, unexpected onset of a health condition that requires immediate medical treatment and/or operation. For example, a cardiopulmonary arrest is an abrupt loss of heart function and if immediate action is not taken, it can prove fatal. In-hospital cardiopulmonary arrest is common and associated with a high mortality rate. When a sudden cardiopulmonary event is discovered to have occurred to a patient in a hospital, a series of actions take place in an attempt to resuscitate that person. Optimally, a response team (referred to conventionally as a code blue response team) consisting of nurses, supervisors, respiratory personnel, technicians and a physician who, preferably, is well-versed in advanced cardiac life support (ACLS) procedures, is summoned to undertake the resuscitation procedures.

Advanced cardiac life support (ACLS) refers to a set of clinical guidelines for the primary treatment of cardiac arrest, stroke, myocardial infarction, and other life-threatening cardiovascular emergencies. The guideline is published by the American Heart Association and the International Liaison Committee of the Resuscitation. The current ACLS guidelines are set into several groups of “algorithms” a set of instructions that are followed to standardize treatment and increase its effectiveness. These algorithms usually come in the form of a flowchart, incorporating ‘yes/no’ type decisions, making the algorithm easier to memorize.

Under emergency conditions, expeditious access to medicines and equipment is crucial. The crash cart was an important invention by the ECRI Institute that helped convey and dispense emergency medication/equipment at the site of medical/surgical emergency for life support protocols (ACLS/ALS) to potentially save someone's life. The cart, also known as a code cart or crash trolley, carries the equipment for cardiopulmonary resuscitation and other medical supplies while also functioning as a support litter for the patient. The cart is typically a set of trays/drawers/shelves on wheels.

Although the response team consisting of nurses, supervisors, respiratory personnel, technicians and a physician should be well-versed in advanced cardiac life support (ACLS) procedures. Most nursing personnel who work on a typical hospital floor are not required to learn or be certified in ACLS. Furthermore, most physicians outside of an intensive care unit (ICU) or emergency room (ER) do not use the ACLS protocols often enough to retain the precise knowledge of the many medications and specific timing sequences involved in a code blue situation. ACLC cards may not be handy in an emergency or updated protocol may not be available. Apparatus are known in the art that overcomes the above problem, for ex., For U.S. Pub. Application 20080125821 discloses a hospital crash cart incorporating an ACLS method and apparatus operable to guide responders, via audible prompts and visual cues, through the proper procedures to be applied to a patient during a cardiac arrest. The apparatus is in the form of a computer with a display fixed to the crash cart.

Hereinafter, the phrase “cardiac pulmonary resuscitation” is also referred to as a procedure. The term “response team” used hereinafter connotes any medical team charged with performing the cardiac pulmonary resuscitation in a hospital. The term “attendant” used hereinafter connotes a member of the response team and includes nurses, supervisors, respiratory personnel, technicians and a physician.

What is needed is a cart that provides all of the tools, patient analysis and instructions to provide cardio recitation, the intelligent code cart disclosed in this document provides the solution.

### BACKGROUND OF THE INVENTION

The present invention relates to a code cart, and in particular, relates to a system and method for handing cardiac pulmonary resuscitation. Known apparatus suffers from one or more drawbacks. The major drawback of the known apparatus is that they require to be manually set by the response team. The response team generally interacts with the apparatus through input means, such as touch screen, mouse, keypad and the like. Firstly, details of the patient are entered and then the required ACLS protocol is determined. The apparats typically function as a digital version of the ACLS guidelines. The team can refer to the desired information from the apparatus like a normal computer. A need is appreciated for an automated system that requires minimum input from the response team and can assist a response team in the procedure of cardiopulmonary resuscitation.

### SUMMARY OF THE INVENTION

It is an object of the intelligent code cart that is directed to an intelligent code cart that could assist the response team in performing cardiopulmonary resuscitation in a hospital or in the field. This can start with a vision system for scanning wristbands on patients or barcodes on drugs or drugs that are removed from the cart for verification before being administered. This integration ensures that the correct equipment and medication are administered, reducing errors and increasing efficiency. The vision system can be used to develop a more intuitive user interface for healthcare providers, with features like gesture control or automatic adjustment to user preferences, enhancing the overall usability of the DataSyteAiCLS.

Another objective of the intelligent code cart to provide provisions conveying and dispensing of medical supplies by directing a care giver to a specific drawer and location within the cart along with the amount of medication that is needed with audio and or video direction with inventory control and re-ordering. This includes retrieving patent diagnostic/monitoring data from the patient monitoring system.

Still another objective of the intelligent code cart is to reduces human error and knowledge gaps during cardiopulmonary resuscitation by monitoring when compressions are made and the number of compressions with audio/video instructions. This can be from camera observation or audio/video demonstration or prompting. There can also be visible and audible alarms appropriate to various unsafe conditions.

Still another objective of the intelligent code cart to enhances the efficiency of the response team in cardiopulmonary resuscitation by providing a link between the cart and distal medical professionals for seamless integration and communication including procedures performed and vital signs as treatment is performed and simulated training of ACLS protocols. There can also be a recording of the procedure for later analysis and training.

Still another objective of the intelligent code cart to provides for the calculation of doses based upon the physical size, age, gender, age and known prior drugs that were ad mistered.

Still another objective of the intelligent code cart to requires minimum input from the response team during cardiopulmonary resuscitation because the cart can view interaction with a patient, track and record progress by video camera, audio recording of the environment in real time for future reference and to ensure cleanliness of the cart. DataSyteAiCLS can understand and interpret spoken instructions from the response team, allowing for more seamless communication and reducing the need for manual input.

Still another objective of the intelligent code cart could retrieve patient records from a database or the web and as well update the patient record with the details of the intervention for inclusion in the patient records. Integration with hospital systems such as electronic health records (EHR) and medical devices can be enhanced. Sensors can communicate with EHRs to provide real-time updates on cart usage, and machine vision can facilitate interaction with other medical devices for coordinated care. DataSyteAiCLS's cameras can transmit live images or videos to doctors located elsewhere, facilitating remote diagnosis and treatment advice.

Still another objective of the intelligent code cart is to aid the response team in locating medicine and equipment arranged within the apparatus. This can be with screen images of a particular drawer, extending a drawer, lighting a specific cavity within a drawer etc.

It is still another object of the intelligent code cart to use artificial intelligence in the DataSyteAiCLS' system to allow for faster and more accurate identification of the appropriate ACLS protocols based on the patient's condition. These algorithms also improve real-time analysis and feedback during resuscitation procedures, leading to better patient outcomes.

It is still another object of the intelligent code cart to include AI-powered predictive analytics that can be used to forecast potential complications or changes in a patient's condition during a resuscitation procedure. This can help the response team make informed decisions and adjust their approach proactively, resulting in improved patient care.

It is still another object of the intelligent code cart to include a vision system with sensors that allows the cart to autonomously navigate through busy hospital corridors and rooms. By processing visual data in real-time, these cameras help the cart avoid obstacles, ensuring smooth and safe transport of medical supplies.

Various objects, features, aspects, and advantages of the present invention will become more apparent from the following detailed description of preferred embodiments of the invention, along with the accompanying drawings in which like numerals represent like components.

### BRIEF DESCRIPTION OF DRAWINGS

FIG. 1 is a pictorial diagram showing different components of the intelligent code cart.

FIG. 2 is a pictorial diagram showing the intelligent code cart in communication with patient records and patient monitoring system.

FIG. 3 shows a perspective view of the intelligent code cart in another preferred embodiment.

FIG. 4 shows a perspective view of the intelligent code cart in another preferred embodiment.

FIG. 5 shows a flow chart of using a deployed intelligent code.

### DETAILED DESCRIPTION OF THE INVENTION

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated. The terminology used herein is for the purpose of describing particular embodiments only and is not intended to be limiting of the technology. As used herein, the singular forms “a,” “an,” and “the” are intended to include the plural forms as well, unless the context clearly indicates otherwise.

It will be further understood that the terms “comprises,” “comprising,” “includes,” and/or “including,” when used in this specification, specify the presence of stated features, integers, steps, operations, elements, and/or components, but do not preclude the presence or addition of one or more other features, integers, steps, operations, elements, components, and/or groups thereof. It will be understood that like or analogous elements and/or components, referred to herein, may be identified throughout the drawings with like reference characters.

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention but is merely representative of various embodiments of the invention. The illustrated embodiments of the invention will be best understood by reference to the drawings, wherein like parts are designated by like numerals throughout.

### ITEM NUMBERS AND DESCRIPTION

| | |
|---|---|
| 100 intelligent code cart | 102 intelligent code cart |
| 110 cart | 111 right side cart |
| 112 left side cart | 115 top platform |
| 120 bottom platform | 120 bottom platform |
| 124 drawer | 125 drawer(s) |
| 126 drawer | 127 medicine |
| 130 castor wheel(s) | 135 label |
| 140 LED indicator | 145 scanner |
| 150 control unit | 151 antenna |
| 155 input devices | 160 speaker(s) |
| 165 monitor | 166 right monitor |
| 167 left monitor | 170 camera(s) |
| 175 patient record database | 180 patient monitoring system |
| 200 leg(s) | 210 pole |
| 215 hook(s) | 220 arm |
| 222 propeller arm | 225 rotor |
| 230 camera | 240 robot arm |
| 300 flow chart | 302 cart deployed |
| 310 monitor patient | 312 outside communication |
| 314 initiate video | 320 open drawer |
| 322 illuminate location | 324 detect removal |
| 330 scan medication | 340 play use video |
| 345 videos | 350 monitor results |

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated. The terminology used herein is for the purpose of describing particular embodiments only and is not intended to be limiting of the technology. As used herein, the singular forms “a,” “an,” and “the” are intended to include the plural forms as well, unless the context clearly indicates otherwise.

It will be further understood that the terms “comprises,” “comprising,” “includes,” and/or “including,” when used in this specification, specify the presence of stated features, integers, steps, operations, elements, and/or components, but do not preclude the presence or addition of one or more other features, integers, steps, operations, elements, components, and/or groups thereof. It will be understood that like or analogous elements and/or components, referred to herein, may be identified throughout the drawings with like reference characters.

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

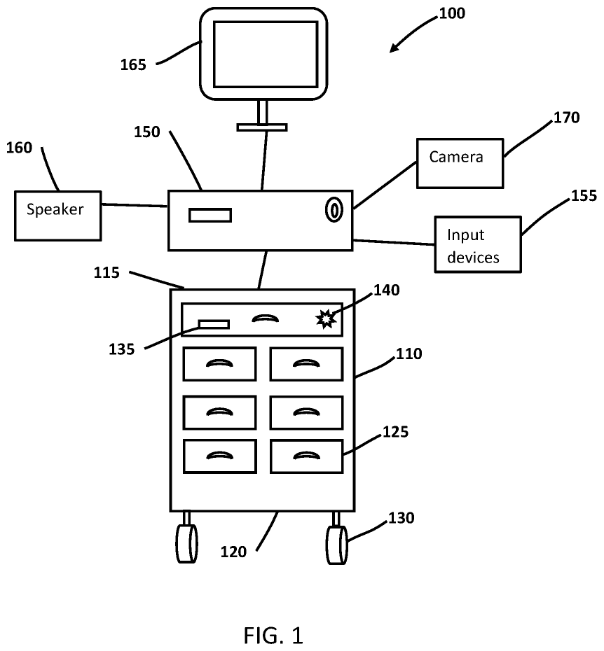

Referring to FIG. 1, which is a pictorial diagram showing different components of the intelligent code cart 100 in accordance with an embodiment of the present invention. As shown in FIG. 1, the intelligent code cart comprises a cart 110 for conveying and dispensing medical supplies and equipment. The structure and functioning of the cart 110 are known in the art for supplying and dispensing of medical supplies for a procedure in a hospital. Cart 110 includes a top platform 115, a bottom platform 120 and a central storage section intermediately positioned between the top platform 115 and the bottom platform 120. Multiple drawers and shelves 125 are provided in the central storage section. Preferably, the drawers 125 are organized according to the ACLS guidelines. The contents of the cart 110 are organized in a manner which reflects current ACLS guidelines drawer by drawer, in a left-to-right fashion. Equipment includes Monitor/defibrillators, suction devices, and bag valve masks (BVMs) of different sizes. The defibrillator can be positioned on the top platform 115 or mounted on the side of the cart 110. The cart 110 is supported on four or more castor wheels 130 mounted at underside of the bottom platform 120. Each drawer/shelf 125 could have a label 135 printed thereon, for example, Arabic numerals could be printed on the front face of each of the drawers 125. These labels 135 can be used to identify the drawer, for example, a particular medicine is stored in the 10th drawer. The 10th drawer can be quickly identified by the label printed thereon. Suitable handles coupled to the drawers help to slide open the drawer. A list of items contained in a drawer could also be provided on the front face of the respective drawer. Such a list could be a printed list pasted on the drawer or an LCD. Further, an LED indicator 140 can be provided on each of the drawers 125 and the LED indicator 140 is electrically connected to a control unit 150. The LED indicator 140 functions to indicate the respective drawer by blinking.

The control unit 150 shown in FIG. 1 comprises a processor and a memory enclosed in a cabinet. Control unit 150 can be installed in the central storage section of the cart 110 itself or can be positioned over the top of the upper platform 115 of the cart 110. The control unit 150 is programmed with the ACLS guidelines including the algorithms. The programming is such that the control unit 150 can analyze the ACLS guideline and provide visible and audible instructions to the response team in performing the cardiopulmonary resuscitation. In another embodiment, control unit 150 can also be configured to connect with the medical equipment contained in cart 110. In one case, the control unit 150 is connected to a blood pressure monitor, pulse oximeter and EKG. Also, control unit 150 can be connected to any patient monitoring system used in the procedure. Control unit 150 in a preferred embodiment can be configured to retrieve the readings from the medical equipment.

In another embodiment, the control unit 150 can also be coupled to one or more of the drawers 125 of the cart 110. One or more of the drawers 125 can be configured with an electric motor to slide open the drawer 125. The electric motor can be triggered by the control unit 150 causing the drawer 125 to pop out. For example, the drawer containing an intended medicine, to be given to the patient, can be opened by control unit 150. Alternatively, the control unit 150 can also trigger the LED indicator 140 on the drawers 125 to indicate the drawer which has the intended medicine.

In another embodiment, control unit 150 can also be configured with inventory management software. Control unit 150 can maintain a running inventory of the contents of the cart 110. The inventory management software can retrieve data from multiple sensors coupled to the cart. The sensors configured to track the withdrawal of the medicines, and the medical equipment form the cart 110. In one embodiment, RFID tags can be coupled to the drawers 125 which can detect the withdrawal of medicines from the drawers 125. This data related to the withdrawal of medicines can be populated in the inventory management software. In another embodiment, an RFID reader can be coupled to the cart 110, such that an attendant withdrawing the medicine from a drawer can scan the medicine with the RFID reader. The same can then be updated in the inventory management software. Besides managing the inventory, control unit 150 can also track the interventions given to the patient based on the medicine withdrawn from the cart 110.

Further shown in FIG. 1 is that the control unit 150 is connected to one or more input devices 155. Input devices 155 can be used to program and instruct the control unit 150. In one case, the input devices include a microphone. The microphone can be used to give voice commands to the control unit 150, for example, an attendant can orally ask the intelligent code cart 100 about the dose of epinephrine to be administered to the patient by saying “what is the dose of epinephrine?” Control unit 150 can analyze the verbal inputs using one or more known algorithms. The functioning of such algorithms/software is known to a skilled person for interpreting voice data. Examples of such algorithms include Siri, Alexa, and Cortana. To make the interpretation of voice commands more accurate, control unit 150 can be configured to respond to only limited vocabulary. The response team can learn the vocabulary of intelligent code cart 100 to verbally interact with the intelligent code cart 100. For example, “ok cart” could trigger the voice input software of the control unit 110. Thereafter, another voice command can be given to the control unit 150, for example, “dose of epinephrine.”

In one case the input means can include a mouse, a keyboard, a touch interface and the like. Different kinds of input devices for use with computers are known in the art and such input devices are within the scope of the present invention. Further can be seen in FIG. 1 that the control unit 150 is connected to a speaker 160. The intelligent code cart 100 can broadcast voice instructions and alerts to the response team through one or more speakers 160.

A display 165 is shown to be coupled with the control unit 150. Display 165 can be a monitor, such as an LCD monitor. The display can be mounted over the upper side of the top platform 115. Display 165 can show graphical instruction, prompts, alerts, and other information to the response team. Also, the display can be configured with a touch interface for interacting with the intelligent code cart 100. The structure and functioning of the tough interfaces coupled to a display are known to a skilled person for interacting with a computer.

The intelligent code cart 100 further comprises one or more cameras 170 connected to the control unit 150. The camera 170 can be mounted over the upper side of the top platform 115 or side of the cart 110. Preferably, the camera 170 can be coupled to the cart 110 through a revolving arm which allows the camera 172 to be adjusted in an intended direction. Control unit 150 can adjust the positioning of the camera 170 to focus on the procedure. Thus, camera 170 can automatically position itself in the intended direction irrespective of the positioning of the intelligent code cart 100. Control unit 150 can also be configured to record the procedure and store the recordings in the memory of the control unit 150. The recordings can be useful for any analysis or training purposes. Furthermore, control unit 150 can be configured with an image recognition algorithm to recognize the actions and gestures of one or more attendants. For example, control unit 150 can recognize that an attendant is giving an injection to the patient or another attendant performing cardiac compressions to the patient.

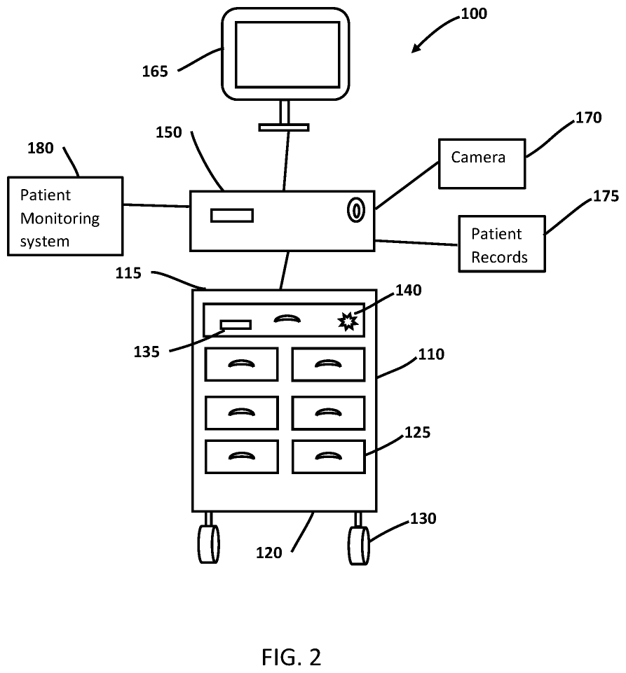

FIG. 2 is a pictorial diagram showing the intelligent code cart 100 connected to a patient record database 175 and a patient monitoring system 180. The patient record database 175 contains records of the patients, wherein each record has an identifier. Control unit 150 can automatically retrieve a record of the patent undergoing cardiopulmonary resuscitation. Moreover, the record of the patient could also be updated with details of the procedure and any intervention given to the patient. This prevents any gaps or omissions in the patient record. In one embodiment, the control unit 150 can also be configured to retrieve readings from a patient monitoring system, for example, the control unit can retrieve the data from ECG, defibrillators, heart rate monitors, oximeters, etc.

In one embodiment, the intelligent code cart 100 of the present invention can be further equipped with simulation training software. The software has a multi-functional simulation training module, which can be used for routine training of the attendants and other hospital staff. So, the response team can get used to the application of intelligent code cart 100 and achieve clinical re-education and training.

In another embodiment, the present invention is directed to a method for assisting a response team in performing cardiopulmonary resuscitation. When a patient reports signs of cardiopulmonary arrest, such as cardiac pain, difficulty in breathing, etc. The response team can be immediately activated to handle the emergency. Meanwhile, the intelligent code cart 100 of the present invention can also be activated. The intelligent code cart 100 on activation can try to identify the patient reporting signs of cardiopulmonary arrest. In case the record of the patient is available in the patient records 175, the intelligent code cart 100 can retrieve the said record which may be having details of the patient, such as name, weight, etc. The intelligent code cart 100 could not automatically identify the patient, a prompt can be generated by the intelligent code cart 100 and an attendant can input the patient id using the keyboard 155 connected to the control unit 150.

The control unit 150 can then retrieve the patient record linked to the entered patient id. Still, if the patient record could not be found by the intelligent code cart 100, the intelligent code cart 100 may prompt the attendant to enter the basic details of the patient, such as the weight of the patient. The weight of the patient can be used to calculate the dose of medicine to be administered to the patient. Firstly, the response team can determine the blood pressure and optionally other parameters, such as pulse and blood oxygen level. The intelligent code cart 100 can retrieve the above determinations. Alternatively, the same could be fed into the intelligent code cart 100 by any attendant. Moreover, other signs of the patient can also be fed to the intelligent code cart 100 by the attendant; for example, the patient is having difficulty in breathing.

Thereafter, the intelligent code cart 100 can trigger the cameras 170 to capture the procedure. Based on the observation fed by the attendant, the intelligent code cart 100 can display a particular ACLS protocol to the response team to be followed. Alternatively, the intelligent code cart 100 can provide a list of ACLS protocols most suited to the present situation. The response team can then select the intended ACLS protocol from the list. The intelligent code cart 100 can then initiate the ACLS protocol by providing both verbal and graphical instructions to the response team. The intelligent code cart 100 can calculate the dose of intervention to be given to the patient and instruct the response team to administer the determined dose of the intervention. The intelligent code cart 100 can indicate the location of the medicine by blinking the indicator 140 on the drawer containing the intended medicine. Alternatively, the intelligent code cart 100 can pop open the drawer containing the intended medicine. This prevents any wastage of time in administering the intervention.

In one embodiment, intelligent code cart 100 can track the procedure and actions of the response team. The intelligent code cart 100 through the images of the procedure captured by the camera 170 can recognize the actions and gestures of the attendants. Moreover, this data can be corroborated with other data, for example, data from sensors coupled to the drawers and patient monitoring system. In one case, the withdrawal of medicine from the drawers as detected by the sensor coupled to the drawer can be corroborated with the action of the attendant administering the intervention to the patient. Moreover, the effect of the intervention can be determined by the intelligent code cart 100 through medical equipment or patient monitoring system. Alternatively, the same could be fed to the intelligent code cart 100 by an attendant. Thus, intelligent code cart 100 can track the whole transaction of instructing the administration of an intervention, withdrawal of medicine from the cart and administration of intervention to the patient by the attendant.

The intelligent code cart 100 through the above-tracked details, can identify any omission of a step of a protocol or if the response team is not following the protocol correctly. Any observation is made by intelligent code cart 100, the intelligent code cart 100 can warn the response team with details of the omission or any errors in following the protocol. Alerts, both graphical and audio, can be broadcasted to the response team. The details of the interventions given to the patient can then be updated in the patient record by the intelligent code cart 100. Moreover, the recordings of the procedure can be stored for later analysis and training.

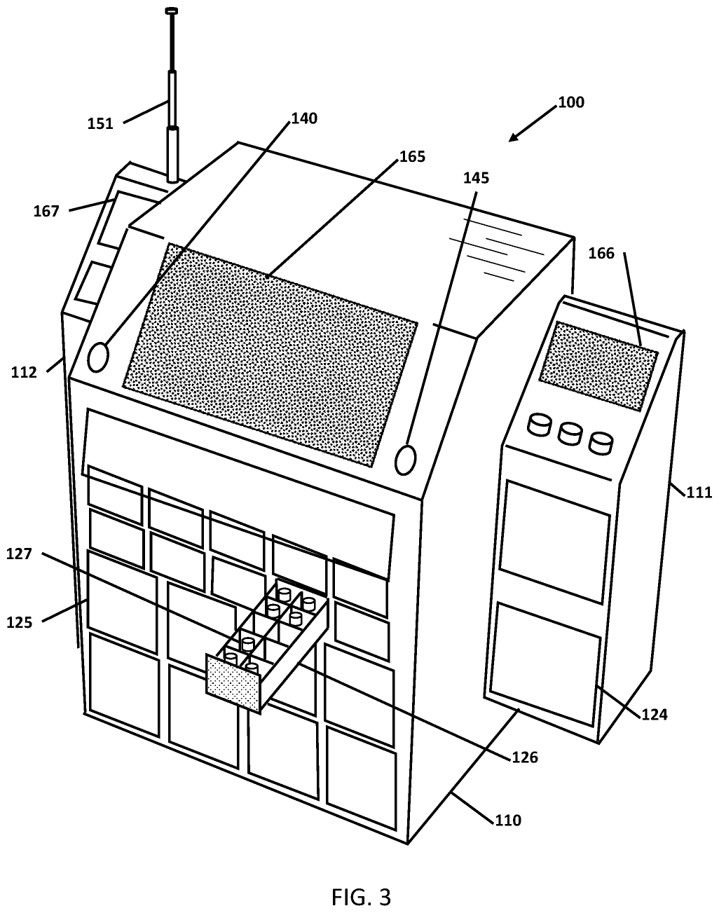

FIG. 3 shows a perspective view of the intelligent code cart 100 in another preferred embodiment with a central monitor 165 for viewing instructional videos or communication with other personnel. This embodiment shows the cart 110 with a right-side cart 111 and a left side cart 112. The side carts can include additional drawers with specific features along with their own right monitor 166 or left monitor 167 that can display additional or supplemental information. In this figure the left side cart 112 is equipped with an antenna 151 for wireless communications to paramedics, hospital, Artificial Intelligence (AI) network. The intelligent code cart 100 can have larger 125 or smaller drawers to accommodate a variety of tools, such as a defibrillator in a drawer 124 or medicines 127 in a drawer 126. The main cart is shown with an LED indicator 140 and a scanner 145 that can read bar codes to verify that the proper medication or medicine 127 is being used. The intelligent code cart 100 can open a drawer 125 and illuminate a specific medication. The drawer may further detect that the proper medication is removed and may further include a scale to determine if the approximate weight of the medication has changed when administered. Billing can be sent to the hospital with point of care billing as the procedure is being performed and medication/equipment I being used.

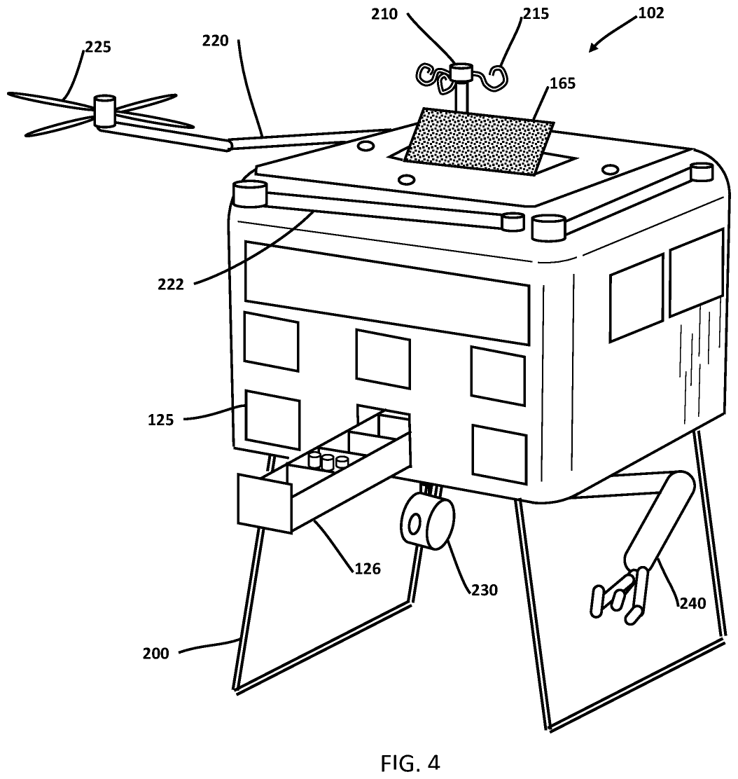

FIG. 4 shows a perspective view of the intelligent code cart 102 in another preferred embodiment. This embodiment is equipped with deployable propeller arm 220 with rotor(s) 225. One arm 220 is shown expanded with at least another propeller arm 222 shown in a folded or retracted orientation. This figure also shows the intelligent code cart 102 with one or more robot arm(s) 240 that allows the intelligent code cart 102 to perform medical procedures either autonomously or under control from distal medical personnel. This figure also includes a pole 210 with hook(s) 215 for hanging bags of medication or other equipment. The bottom of the intelligent code cart 102 has a camera 215 and supporting legs 200. This intelligent code cart 102 can be deployed from a hospital or vehicle to a distal location to provide medical assistance with or without a trained medical person. Monitor 165 can show instructions or allow for real-time conversations with a person or artificial intelligence avatar. Where one or more specific drawer(s) 125, 126 can be opened or unlocked to allow access to medication(s).

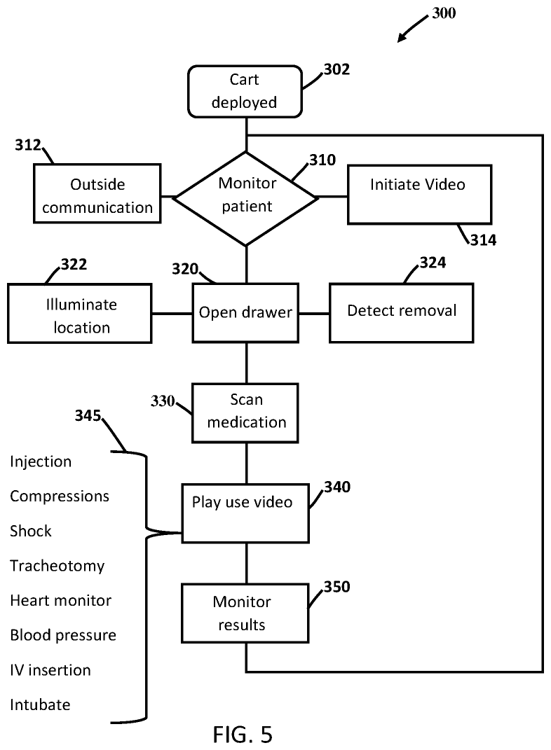

FIG. 5 shows a flow chart 300 of using a deployed intelligent code. From the top of the flow chart 300 the cart is deployed 302. The deployment can be by rolling the cart to a person, rolling a person to a cart or flying the cart to the person. When the cart is initialized, it uses sensors to monitor the patient, or the vital signs of the patient can be entered manually. Outside communication 312 can be initiated along with a video 314 link using a distal person or avatar that is linked to an artificial intelligence database. One or more drawers 320 can be opened to exposed medication or equipment. The specific medication or equipment can be illuminated 322 and the cart can detect removal 324 that will progress the video to the next stage of a procedure. The cart can scan the medication 330 or equipment using a bar code, QR code or another identifier. Again, this may progress the video 340 to another playing of how to use the equipment or medication. The video 345 can instruct use of medication or equipment, including, but not limited to, injection, compressions, shock, tracheotomy, heart monitor, blood pressure, IV insertion or intubate. After, and while, providing medical support, the cart can monitor the results 350 of the patient and may result in administering additional equipment or medication.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

It is further contemplated that the cart includes a DataSyteAI system that mimics the cognitive and analytical processes of top-tier professionals, specifically medical practitioners and cross-industry experts. The primary objective of this technology is to enhance diagnostic precision and decision-making in healthcare, extend the AI's adaptability to cross-industries for tasks such as risk assessment, manufacturing optimization, and professional task mimicry.

The DataSyteAI employs a multi-layered neural network architecture combined with real-time reinforcement learning and deep learning algorithms to analyze, synthesize, and replicate human expertise. Key components include data input layer with structured and unstructured data inputs, including patient medical records, imaging, and industrial datasets.

It would further have a learning module for supervised and unsupervised machine learning models as well as predictive analytics and training datasets curated from leading experts' decisions.

A cognitive mimicry engine would have trained models emulate top professionals' cognitive processes that employs benchmarked accuracy scores to match professional expertise.

A decision output would generate diagnoses, actionable insights, and recommendations for end users.

The AI undergoes continuous algorithmic refinement, enabling it to adapt, learn, and match real-world expert decision-making across fields.

The DataSyteAI is designed as a modular AI system with inputs for EHR (Electronic Health Records), DICOM imaging (MRI, CT scans), and large datasets and cross-industry inputs like manufacturing metrics or financial reports. There is a processing pipeline for preprocessing data normalization and feature extraction and neural network layers for processes data using deep learning models.

The DataSyteAI has a mimicry algorithm that is based on benchmarked datasets of leading professionals' outputs that uses historical decisions to replicate clinical reasoning.

There are key features for diagnostic mimicry for medical professionals when analyzing an MRI scan, the AI replicates the cognitive workflow of a radiologist to determine potential pathologies.

Decision validation metrics use outputs a “Mimicry Score,” indicating how closely the AI output aligns with expert decisions. Cross-industry adaptability uses retraining modules that allows the AI to extend to industries such as logistics, financial auditing, and manufacturing.

Core algorithmic DataSyteAiCLS framework operates with data Ingestion that collect multi-modal data streams from various sources with feature extraction and preprocessing to identify key patterns and features relevant to each professional task.

Neural network computation has multi-layer convolutional neural networks (CNNs) for image-based tasks for recurrent Neural Networks (RNNs) for sequential decision-making processes.

A mimicry module reinforcement learning fine-tunes the AI to align its output with benchmark decisions from top professionals.

Output generation results include diagnosis suggestions, actionable insights and performance predictions (cross-industry).

Thus, specific embodiments of intelligent code cart have been disclosed. It should be apparent, however, to those skilled in the art that many more modifications besides those described are possible without departing from the inventive concepts herein. The inventive subject matter, therefore, is not to be restricted except in the spirit of the appended claims.

1. A method of operating an intelligent code cart, the method comprising:
providing the intelligent code cart comprising of a control unit having a processor, memory, a vocabulary;
a central storage section with at least two drawers that are configured to enclose at least one medicine and at least one piece of medical equipment;
a plurality of indicators coupled to one or more of said at least two drawers that is triggered to open or unlock by said control unit;
at least one sensor coupled to at least one of said at least two drawers that is configured to detect withdrawal of a medicine or medical equipment;
at least one camera that is configured to autonomously position itself;
a speaker, a microphone and at least one monitor connected to said control unit;
determining at least a first medical procedure from manual input or from at least one medical sensor;
said at least a first medical procedure opens or unlocks at least one of said at least two drawers;
illuminating a location of said medication or said equipment and prompting instructions on said at least one monitor;
receiving a signal from at least one of said plurality of indicators and prompting a second instruction on said at least one monitor, and
further monitoring a result of said first medical procedure and determining if a second medical procedure is required.

2. The method of claim 1, wherein the first patient details are retrieved by the intelligent code cart from a patient record database.

3. The method of claim 1, wherein the first patient details are received, by the intelligent code cart, from the one or more attendants.

4. The method of claim 1, wherein the first patient details comprise weight of the patient, and the method further comprises a step of calculating dose of an intervention based on the weight of the patient.

5. The method of claim 1, wherein the step of identifying a standard protocol further comprises steps of: displaying, by the intelligent code cart, to the response team, a list of standard protocols, the list of standard protocols based on the first patient details and observed signs of the patient; and selection, by the response team, of the standard protocol from the list of the standard protocols.

6. The method of claim 1, wherein the standard protocol is based on ACLS guidelines.

7. The method of claim 1, wherein the method further comprises a step of updating a patient record with details of interventions admitted to the patient.

8. The method of claim 1, wherein the method further comprises a step of receiving, by the intelligent code cart, details of withdrawal of an intended medicine from one of the plurality of drawers.

9. The method of claim 8, wherein the method further comprises a step of: receiving, by the intelligent code cart, an action of the administration of the intended medicine to the patient based on analysis of the sequence of images; and corroborating the details of the withdrawal of the intended medicine to the action of the administration of the intended medicine.

10. The method of claim 1, wherein the method further comprises a step of guiding the response team to one drawer of the plurality of the drawers by blinking an indicator coupled to the one drawer, the one drawer containing a medicine to be administered to the patient.

11. The method of claim 1, wherein the method further comprises a step of opening one drawer of the plurality of the drawers by the control unit, wherein the one drawer is having a medicine instructed by the intelligent code cart for administration to the patient.

12. The method of claim 1, wherein the step of observing any error in following the standard protocol is based on comparison of the actions of the response team with the standard protocol.

13. The method of claim 1, wherein the plurality of images is a video, and the method further comprise a step of storing the video in the memory of the control unit.

14. The method of claim 1, wherein the step-by-step instructions are displayed on the display and broadcasted through the speaker.

15. The method of claim 1, wherein the method further comprises a step of retrieving, by the control unit, data from the medical equipment, the data related to a condition of the patient determined by the medical device.

16. An intelligent code cart comprising:
a control unit configured with a vocabulary;
a central storage section, the central storage section comprises at least one camera, a speaker, an input device, a plurality of drawers, the plurality of drawers coupled to the control unit;
one or more medical equipment, the one or more medical equipment mounted to the central storage section, the one or more medical equipment in electrical communication with the control unit;
a plurality of indicators coupled to one or more of the plurality of drawers, the plurality of indicators configured to be triggered by the control unit to blink; one or more sensors coupled to the one or more of the plurality of drawers, the one or more sensors configured to detect withdrawal of a medicine from a drawer;
the at least one camera coupled to the central storage unit, the at least one camera being configures to lock to a view while said intelligent code cart is moving;
a display mounted on top of the central storage section; the display coupled to the control unit; a speaker coupled to the control unit;
and one or more input devices coupled to the control unit; wherein the control unit is configured to: detect withdrawal of the medicine from the drawer, capturing a sequence of images of the response team by the one or more cameras, and recognize actions and gestures of the response team from the sequence of images.

17. The intelligent code cart of claim 16, wherein the intelligent code cart further comprises a motor coupled to the plurality of drawers, wherein the motor can be triggered by the control unit to open one of the plurality of drawers.

18. The intelligent code cart of claim 16, wherein the one or more input devices includes a microphone, the controlling unit is configured to interpret voice commands received through the microphone.

19. The intelligent code cart of claim 16, wherein the display has a touch input interface.

20. The intelligent code cart of claim 16, wherein the at least one sensor is an RFID sensor.
